(19) United States
(12) Patent Application Publication
(10) Pub. No.: US 2014/0276485 A1
(43) Pub. Date: Sep. 18, 2014

(54) STRETCH MARK THERAPY AND MOISTURIZING GARMENT

(71) Applicant: Netta Grutman, Woodland Hills, CA (US)

(72) Inventor: Netta Grutman, Woodland Hills, CA (US)

(21) Appl. No.: 13/803,546

(22) Filed: Mar. 14, 2013

(51) Int. Cl. A61M 35/00

(52) U.S. Cl. CPC A61M 35/00
USPC 604/308

(57) ABSTRACT

The present invention is preferably a stretch mark treatment and prevention garment composed of at least two layers. The inner layer(s) is preferably comprised of an essentially non-absorbent synthetic fabric, and the outer layer is preferably comprised of a fashionable synthetic or non-synthetic fabric. The garment of the present invention preferably functions to hold emollient substances onto a wearer's skin and to restrict those substances from seeping out of the garment and possibly staining the wearer's outer garments. The garment provides continuous moisturizing to a wearer's skin without the emollients being absorbed other garments worn by the wearer.

### FIELD OF INVENTION

This invention relates generally to apparel, and more specifically, to a garment or undergarment worn around a person's abdomen, chest, lower torso, legs, or hips for the prevention and treatment of scars or stretch marks and for moisturizing skin.

### BACKGROUND

For centuries, many people, such as pregnant mothers, have suffered from stretch marks. Stretch marks are a form of scarring caused by the tearing of elastic fibers within the dermis or layer of skin. Elastic fibers in the dermis tear when rapid stretching of the skin occurs during short periods of substantial growth or weight change. Stretch marks are typically developed on the abdomen, chest, lower torso, legs or buttocks of women while undergoing puberty and/or pregnancy. Many women suffering stretch marks generally apply balms, lotions or topical ointments to their skin as a preventative measure against stretch marks. Moisturizers, including alcohol-based moisturizers, are applied and absorbed quickly by the skin or evaporate, which means these moisturizers do not provide long lasting moisturizing and therapeutic effects. Examples of beneficial lotions and moisturizers include Vitamin E oil or other natural waxes and oils that do not readily evaporate when applied to a user's skin. This results in the problem of the moisturizers being absorbed by various types of fabrics that are donned by the user which means these moisturizers do not provide long lasting moisturizing and therapeutic effects.

Various references disclose garments that help women prevent and treat stretch marks. For example, U.S. Patent Application Publication No. 2011/0230119, filed by Thompson discloses garments that utilize constant adherent traction with a wearer's skin to provide added support and stability to the wearer's dermal layer while providing the treatment of stretch marks. While this reference discloses using a garment to treat and prevent stretch marks, it neither discloses the use of any moisturizing agent for the treatment of stretch marks nor does it disclose a garment that does not allow the moisturizing agents applied to the wearer's skin to be absorbed by the garment itself or the wearer's other clothing.

Other references include U.S. Pat. No. 8,084,051, issued to Dillon, and U.S. Pat. No. 2,601,851, issued to Jones, both of which disclose garments that keep anti-scaring agents in constant contact with a wearer's skin. The Dillon reference discloses a garment with a composite sheet composed of a layer of textile fabric and a layer of silicone elastomer that is applied to an area of the body for scar treatment. Importantly, however, this reference lacks a layer of fabric that is designed to be non-absorbent of alcohol-free moisturizers. The Jones reference discloses a medicated garment for holding medicine against the skin for treatment of skin ailments. Notably, however, the Jones reference does not disclose a garment that limits absorption of the moisturizing agents applied to the wearer's skin.

Although the above references disclose various garments for the treatment and prevention of scars and stretch marks, none of the references is directed towards a garment that includes fabric designed to provide constant contact and hold the moisturizer to a user's skin while restricting any seepage or absorption of the moisturizer into or onto a user's clothing.

Therefore, what is needed is a garment that provides continuous contact of moisturizer to the skin while, at the same time, restricting the moisturizer from being absorbed by outer clothing.

### BRIEF SUMMARY OF THE INVENTION

To minimize the limitations in the prior art, and to minimize other limitations that will become apparent upon reading and understanding the present specification, the present invention discloses a stretch mark treatment and prevention garment composed of several layers.

One embodiment of the present invention is a stretch mark therapy garment comprising: at least one inner layer; and at least one outer layer. The at least one inner layer is attached to the at least one outer layer, such that the at least one inner layer is configured to substantially contact the skin of the wearer. The at least one inner layer is constructed of one or more essentially non-absorbent materials. An emollient is applied to a portion of the skin of the wearer at least where the garment is worn by the wearer. The at least one inner layer is configured to hold the emollient against the skin of the user, such that the at least one inner layer substantially restricts the emollient from being absorbed by the garment, substantially restricts the emollient from contacting any other article of clothing worn by the user, and substantially restricts said emollient from seeping outside of said garment. Preferably, the emollient is a moisturizing substance. Preferably, the at least one inner layer is constructed of one or more synthetic fiber materials. Preferably, the at least one inner layer is constructed from one or more materials selected from the group of materials consisting of: nylon, spandex, and polyester. Preferably, the at least one outer layer is constructed from one or more materials selected from the group of materials consisting of: spandex and cotton. Preferably, the at least one inner layer is comprised of a first inner layer and a second inner layer. Preferably, the first inner layer is configured to keep the emollient in substantial constant contact with the skin of the user. Preferably, the stretch mark therapy garment is configured to be worn around an abdomen of the user. Preferably, the stretch mark therapy garment is selected from the group of garments consisting of: a bra, a belly wrap, an undergarment, and a pair of shorts.

Another embodiment of the invention is a stretch mark therapy garment comprising: at least one inner layer; and at least one outer layer. The at least one inner layer is attached to the at least one outer layer, such that the at least one inner layer is configured to contact a skin of a wearer. The at least one inner layer and the at least one outer layer are constructed of one or more essentially non-absorbent materials. An emollient is applied to a portion of the skin of the wearer at least where the garment is worn by the wearer. The at least one inner layer is configured to hold the emollient against the skin of the user. The at least one outer layer substantially restricts the emollient from contacting any other article of clothing worn by the user, and substantially restricts said emollient from seeping outside of said garment. Preferably the emollient is a moisturizing substance. Preferably, the at least one inner layer is constructed of one or more synthetic fiber materials selected from the group of materials consisting of: nylon, spandex, and polyester. Preferably, the at least one outer layer is constructed from spandex. Preferably, the at least one inner layer is comprised of a first inner layer and a second inner layer; wherein the first inner layer is configured to keep the emollient in substantially constant contact with the skin of the user. Preferably, the stretch mark therapy garment is configured to be worn around an abdomen of the user. Preferably, the stretch mark therapy garment is selected from the group of garments consisting of: a bra, a belly wrap, an undergarment, and a pair of shorts.

Another embodiment of the invention is a stretch mark therapy garment comprising: at least one inner layer; at least one middle layer; and at least one outer layer. The at least one inner layer, the at least one middle layer and the at least one outer layer are attached such that the at least one inner layer is configured to contact a skin of a wearer and the at least one middle layer is disposed between the at least one inner layer and the at least one outer layer. The at least one inner layer and the at least one middle layer are constructed of one or more essentially non-absorbent materials. An emollient is applied to a portion of the skin of the wearer at least where the garment is worn by the wearer. The at least one inner layer is configured to hold the emollient against the skin of the user. The at least one middle layer substantially restricts the emollient from being absorbed by the garment, substantially restricts the emollient from contacting any other article of clothing worn by the user, and substantially restricts said emollient from seeping outside of said garment. Preferably, the emollient is a moisturizing substance. Preferably, the at least one inner layer and the at least one middle layer is constructed of one or more synthetic fiber materials selected from the group of materials consisting of: nylon, spandex, and polyester. Preferably, the at least one outer layer is constructed from one or more materials selected from the group of materials consisting of cotton and spandex. Preferably, the stretch mark therapy garment is selected from the group of garments consisting of: a bra, a belly wrap, an undergarment, and a pair of shorts.

It is an object of the present invention to provide a stretch mark treatment garment comprised of several layers; wherein the garment preferably functions as a barrier between a person's outer clothing and the emollient applied on the person's skin for the treatment/prevention of stretch marks or for moisturizing the skin.

It is an object of the present invention to create a garment that provides continuous moisturizing to a person's skin without the possibility of moisturizing substances being absorbed by the person's outer clothing.

It is an object of the present invention to provide a stretch mark therapy garment that takes the form of a bra (sports or regular), a belly wrap, an undergarment, and a pair of shorts

It is an object of the present invention to provide a stretch mark therapy garment that will allow users to apply the garment to their body directly after application of moisturizers to their skin.

It is an object of the present invention to overcome the limitations of the prior art.

These, as well as other components, steps, features, objects, benefits, and advantages, will now become clear from a review of the following detailed description of illustrative embodiments, the accompanying drawings, and the claims.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is a perspective view of one embodiment of the stretch mark therapy garment and shows the garment with three layers, two inner layers and an outer layer.

FIG. 2 is a cross-sectional view of one embodiment of the stretch mark therapy garment.

FIG. 3 is a front view of one embodiment of the stretch mark therapy garment and shows the garment constructed as a belly band.

FIG. 4 is a rear view of one embodiment of the stretch mark therapy garment and shows the stretch mark therapy garment constructed as a bra.

FIG. 5 is a cross-sectional view of one embodiment of the stretch mark therapy garment and shows the stretch mark therapy garment constructed as a pair of shorts.

### DETAILED DESCRIPTION OF THE DRAWINGS

In the following detailed description of the preferred embodiment, reference is made to the accompanying drawings that form a part hereof, and in which is shown, by way of illustration, a specific embodiment in which the invention may be practiced. It is to be understood that other embodiments may be utilized and structural changes may be made without departing from the scope of the present invention.

In the following detailed description of various embodiments of the invention, numerous specific details are set forth in order to provide a thorough understanding of various aspects of one or more embodiments of the invention. However, one or more embodiments of the invention may be practiced without these specific details. In other instances, well-known methods, procedures, and/or components have not been described in detail so as not to unnecessarily obscure aspects of embodiments of the invention.

In the following description, certain terminology is used to describe certain features of one or more embodiments of the invention. For instance, the terms “therapeutic liquid”, “emollient”, “moisturizer”, “lotion”, and “cream”, all refer to liquids or other substances (aqueous and/or oil based) that may be used for the treatment of scars, stretch marks, dry skin, and other skin conditions and disorders, including without limitation, hydrating creams, lotions, body butters, vitamin E oil, lubricants, scar treatment creams, stretch mark creams, oil-based moisturizers, humectants, polyhydroxy alcohols, (e.g., glycerol, butylene glycol, hexylene glycol, propylene glycol, tetraglycol, sorbitol and the like); lactic acid and lactate salts (e.g., sodium or ammonium salts); diols and triols (e.g., hexylene glycol, dihydroxyhexane, hexane triol); aloe vera in any of its forms (e.g., aloe vera gel); sugars and starches; sugar and starch derivatives (e.g., alkoxylated glucose); hyaluronic acid; lactamide monoethanolamine; acetamide monoethanolamine; glycolic acid; and alpha and beta hydroxy acids (e.g. lactic, glycolic salicylic acid).

As used herein, the term “garment” refers to an apparel item which covers some portion of the body, including without limitation: the chest, abdomen, waist, belly, hips, buttocks, thighs, and legs. In various embodiments, the garment may be a bra, a belly wrap, an undergarment, or a pair of shorts

The present invention is preferably a stretch mark treatment and prevention garment composed of two or more layers, but one layer may be used. If only one layer is used, this layer is preferably an essentially non-absorbent layer. The preferred embodiment has three layers. Two of the layers are preferably comprised of essentially non-absorbent synthetic fabric, and the third layer may be comprised of absorbent or essentially non-absorbent synthetic or non-synthetic fabric. The non-absorbent fabric may be a very low absorbent or essentially non-absorbent fabric. The garment of the present invention preferably functions as a barrier between a person's outer clothing and the emollient applied on the person's skin for the treatment and/or prevention of stretch marks or moisturizing of the skin. Preferably, the garment provides continuous moisturizing to a person's skin without the worry of emollients being absorbed by their outer clothing. It should be understood that many other dermatological conditions may be treated by the present invention.

FIG. 1 is a perspective view of one embodiment of the stretch mark therapy garment and shows the garment with three layers, two inner layers and an outer layer. As shown in FIG. 1, the stretch mark therapy garment 100 preferably includes: an inner layer 102; a middle layer (or second inner layer) 104; and an outer layer 106. The inner layer 102 is preferably constructed of one or more essentially non-absorbent materials such as spandex, nylon, polyester, or any combination thereof, but may be constructed out of any essentially non-absorbent material, such as elastane, acrylic, rayon, acetate, oletin, and/or kevlar. The inner layer 102 is preferably used to hold emollients 110, creams, or moisturizers, such as scar treatment creams against a wearer's skin 120. Preferably the inner layer 102 holds the emollient 110 in continuous contact with the skin.

Although only one inner layer is shown, it should be understood that the inner layer, and any of other layers, may be comprised of multiple layers that make up that layer. It should also be understood that the garment may be made up of only two layers, an inner and an outer layer, without deviating from the scope of the invention. It should also be understood that the middle layer may be an additional outer or inner layer.

One of the layers, middle, inner, or outer, preferably acts as a barrier layer that restricts the emollient from being absorbed by any absorbent portion of the garment, restricts the emollient from contacting any other article of clothing worn by the user, and restricts said emollient from seeping outside of said garment. Whichever layer or layers are the barrier layers are preferably constructed out of one or more essentially non-absorbent synthetic materials, such as spandex, nylon, polyester, elastane, acrylic, rayon, acetate, oletin, and/or kevlar. If the inner layer is the barrier layer, the outer layer and/or the middle layer may be constructed out of any synthetic or man-made material, absorbent or essentially non-absorbent, including, but not limited to: cotton, wool, spandex, nylon, polyester, elastane, acrylic, rayon, acetate, oletin, and/or kevlar. If the middle layer is the barrier layer, the outer layer may be constructed out of any synthetic or man-made material, absorbent or essentially non-absorbent, including, but not limited to: cotton, wool, spandex, nylon, polyester, elastane, acrylic, rayon, acetate, oletin, and/or kevlar. The inner layer is preferably essentially non-absorbent. It should also be understood that one or more absorbent layers may be thick enough or absorbent enough to act as a barrier layer. This is not preferred because this more quickly removes the emollient from contact with the skin.

The outer layer 106 is preferably constructed of a fashionable and/or sturdy material such as Lycra®, a type of spandex or elastane, or cotton. Although FIG. 1 shows that the layers are in contiguous contact, it should be understood that they are preferably connected at seams and hems and may not necessarily be fused or attached together in such a literal manner as shown.

The inner layer(s) may be mesh, such that the emollient is contained within the holes of the mesh to provide a comfortable way to continuously hold the emollient in contact with the wearer's skin.

FIG. 2 is a cross-sectional view of one embodiment of the stretch mark therapy garment. As shown in FIG. 2, the stretch mark therapy garment 100 preferably includes: an inner layer 102; a middle layer (or second inner layer) 104; and an outer layer 106. FIG. 2 shows how the garment 100 holds an emollient 110 next to the skin 120 of the wearer.

FIG. 3 is a front view of one embodiment of the stretch mark therapy garment and shows the garment constructed as a belly band. A belly wrap or band is generally a versatile garment that is worn around a user's waist or abdomen. As shown in FIG. 3, the garment 100 is preferably comprised of outer layer 106, inner layers 102, fastener 230, and top and bottom support bands/seams 220 and 222. As shown in FIG. 3, the inner layers 102 are preferably mesh and are constructed out of a synthetic essentially non-absorbent nylon or spandex, which allow the garment to hold an emollient (shown in FIGS. 1 and 2) in contact with a wearer's skin. FIG. 3 shows how the outer layer 106 is preferably spandex, such a Lycra®. FIG. 3 shows how the outer layer may be an essentially non-absorbent layer that restricts the emollient from being absorbed by the garment, restricts the emollient from contacting any other article of clothing worn by the user, and restricts said emollient from seeping outside of said garment. In this manner, the emollient is held against the wearer's skin and is not absorbed into the garment 100 or allowed to escape outside the garment 100

The garment 100 typically works in conjunction with a moisturizer that is applied on the skin or to the inner layer 102, such that, when the belly band garment 100 is worn around a wearer's waist, the emollient is continuously applied. This generally provides users with application of significant amounts of oil and/or moisturizer to regions of the body that might suffer scarring or stretch marks due to pregnancy or weight changes. This also restricts seepage of the oil, cream, and/or moisturizer to the user's outer clothing or garments. By wearing the stretch mark therapy garment 100 under the wearer's outer clothing, the wearer may go about their daily lives while simultaneously enjoy the moisturizing effects of the cream.

FIG. 4 is a rear view of one embodiment of the stretch mark therapy garment and shows the stretch mark therapy garment constructed as a bra. As shown in FIG. 4, the garment 300 is preferably comprised of an outer layer 306, inner layers 302, and bottom support band 322. As shown in FIG. 4, the bottom support band 322 is preferably elastic such that the garment 300 is held in place and the emollient is further contained within an interior of garment 300.

FIG. 5 is a cross-sectional view of one embodiment of the stretch mark therapy garment and shows the stretch mark therapy garment constructed as a pair of shorts. As shown in FIG. 5, the garment may be a bra 300 or a pair of shorts 400.

Unless otherwise stated, all measurements, values, ratings, positions, magnitudes, sizes, locations, and other specifications which are set forth in this specification, including in the claims which follow, are approximate, not exact. They are intended to have a reasonable range which is consistent with the functions to which they relate and with what is customary in the art to which they pertain.

The foregoing description of the preferred embodiment of the invention has been presented for the purposes of illustration and description. While multiple embodiments are disclosed, still other embodiments of the present invention will become apparent to those skilled in the art from the above detailed description, which shows and describes illustrative embodiments of the invention. As will be realized, the invention is capable of modifications in various obvious aspects, all without departing from the spirit and scope of the present invention. Accordingly, the detailed description is to be regarded as illustrative in nature and not restrictive. Also, although not explicitly recited, one or more embodiments of the invention may be practiced in combination or conjunction with one another. Furthermore, the reference or non-reference to a particular embodiment of the invention shall not be interpreted to limit the scope the invention. It is intended that the scope of the invention not be limited by this detailed description, but by the claims and the equivalents to the claims that are appended hereto.

Except as stated immediately above, nothing which has been stated or illustrated is intended or should be interpreted to cause a dedication of any component, step, feature, object, benefit, advantage, or equivalent to the public, regardless of whether it is or is not recited in the claims.

What is claimed is:

1. A moisturizing garment comprising:
at least one inner layer;
wherein said at least one inner layer is constructed of one or more essentially non-absorbent materials; and
wherein an emollient is applied to a portion of said skin of said wearer at least where said garment is worn by said wearer.

2. The garment of claim 1, further comprising:
at least one outer layer;
wherein said at least one inner layer is attached to said at least one outer layer, such that said at least one inner layer is configured to substantially contact the skin of a wearer.

3. The garment of claim 1, wherein said at least one inner layer is configured to hold said emollient against said skin of said user, such that said at least one inner layer substantially restricts said emollient from seeping outside said garment.

4. The garment of claim 3, wherein said emollient is a moisturizing substance.

5. The garment of claim 3, wherein said at least one inner layer is constructed of one or more synthetic fiber materials.

6. The garment of claim 3, wherein said at least one inner layer is constructed from one or more materials selected from the group of materials consisting of: nylon, spandex, and polyester.

7. The garment of claim 3, wherein said at least one outer layer is constructed from one or more materials selected from the group of materials consisting of: spandex and cotton.

8. The garment of claim 3, wherein said at least one inner layer is comprised of a first inner layer and a second inner layer;
wherein said first inner layer is configured to keep said emollient in substantially constant contact with said skin of said user.

9. The stretch mark therapy garment of claim 3, wherein said garment is selected from the group of garments consisting of: a bra, a belly wrap, an undergarment, and a pair of shorts.

10. A moisturizing garment comprising:
at least one inner layer; and
at least one outer layer;
wherein said at least one inner layer is attached to said at least one outer layer, such that said at least one inner layer is configured to contact a skin of a wearer;
wherein said at least one inner layer and said at least one outer layer are constructed of one or more essentially non-absorbent materials;
wherein an emollient is applied to a portion of said skin of said wearer at least where said garment is worn by said wearer;
wherein said at least one inner layer is configured to hold said emollient against said skin of said user;
wherein said at least one outer layer substantially restricts said emollient from seeping outside of said garment.

11. The garment of claim 10, wherein said emollient is a moisturizing substance.

12. The garment of claim 10, wherein said at least one inner layer is constructed of one or more synthetic fiber materials selected from the group of materials consisting of: nylon, spandex, and polyester.

13. The garment of claim 10, wherein said at least one outer layer is constructed from spandex.

14. The garment of claim 10, wherein said at least one inner layer is comprised of a first inner layer and a second inner layer;
wherein said first inner layer is configured to keep said emollient in substantially constant contact with said skin of said user.

15. The garment of claim 10, wherein said stretch mark therapy garment is selected from the group of garments consisting of: a bra, a belly wrap, an undergarment, and a pair of shorts.

16. A moisturizing garment comprising:
at least one inner layer;
at least one middle layer; and
at least one outer layer;
wherein said at least one inner layer, said at least one middle layer and said at least one outer layer are attached such that said at least one inner layer is configured to contact a skin of a wearer and said at least one middle layer is disposed between said at least one inner layer and said at least one outer layer;
wherein said at least one inner layer and said at least one middle layer are constructed of one or more essentially non-absorbent materials;
wherein an emollient is applied to a portion of said skin of said wearer at least where said garment is worn by said wearer;
wherein said at least one inner layer is configured to hold said emollient against said skin of said user;
wherein said at least one middle layer substantially restricts said emollient from seeping outside of said garment.

17. The garment of claim 16, wherein said emollient is a moisturizing substance.

18. The garment of claim 16, wherein said at least one inner layer and said at least one middle layer is constructed of one or more synthetic fiber materials selected from the group of materials consisting of: nylon, spandex, and polyester.

19. The garment of claim 16, wherein said at least one outer layer is constructed from one or more materials selected from the group of materials consisting of: spandex and cotton.

20. The garment of claim 16, wherein said stretch mark therapy garment is selected from the group of garments consisting of: a bra, a belly wrap, an undergarment, and a pair of shorts.
